(19) United States
(12) Patent Application Publication
(10) Pub. No.: US 2004/0082921 A1
(43) Pub. Date: Apr. 29, 2004

(54) Infusion port apparatus of a vascular infusion assembly

(76) Inventor: Cho Ying Chen, Taichung (TW)

Correspondence Address: Cho Ying Chen, P.O. BOX 2103, Taichung, TW

(21) Appl. No.: 10/687,744

(22) Filed: Oct. 20, 2003

(30) Foreign Application Priority Data
Oct. 23, 2002 (TW) 91217086

(51) Int. Cl. A61M 5/00

(52) U.S. Cl. 604/246

(57) ABSTRACT

An infusion port apparatus of a vascular infusion assembly includes a first post longitudinally communicating with a guide post of the vascular infusion assembly and a second post extending from the first post to form a corner and communicating with the first post. A feeder is partially airtightly received in the second post. The feeder includes a hollow shank having a first end extending into and received in the second post, and a second end opposite to the first end of the hollow shank. Multiple holes are defined in the hollow shank near the first end of the hollow shank and laterally communicating with an inner periphery of the hollow shank. A resilient ring is mounted around the hollow shank to selectively close the multiple holes in the hollow shank.

### BACKGROUND OF THE INVENTION

1. Field of the Invention

The present invention relates to an infusion apparatus, and more particularly to an infusion apparatus of a vascular infusion assembly.

Vascular infusion assemblies are common and known in the medical field, so a general background description of them is not provided here. Existing vascular infusion assemblies are problematic in that they typically provide an infusion port that can only be accessed by use of a sharp, such as a needle, to infuse a desired infusate fluid, such as medicine, blood, nutrients, electrolyte solution, anesthesia, muscle relaxants, or other desired infusates. For health, safety and convenience reasons, medical personnel often wish to avoid use of a sharp. Use of a sharp involves a risk of inadvertent self-impalement or the impalement of others, introducing risks of injury and disease for both the user of the sharp and for all others present.

### SUMMARY OF THE INVENTION

The main objective of the present invention is to provide an improved infusion port apparatus of a vascular infusion assembly to which a sharp needle is unnecessary during adding agent into the vascular infusion assembly.

To achieve the objective, the infusion port apparatus in accordance with the present invention includes a first post longitudinally communicating with a guide post of the vascular infusion assembly and a second post extending from the first post to form a corner and communicating with the first post. A feeder is partially airtightly received in the second post. The feeder includes a hollow shank having a first end extending into and received in the second post, and a second end opposite to the first end of the hollow shank. Multiple holes are defined in the hollow shank near the first end of the hollow shank and laterally communicating with an inner periphery of the hollow shank. A resilient ring is mounted around the hollow shank to selectively close the multiple holes in the hollow shank. When adding agent into the vascular infusion assembly, a needle hub of a syringe is inserted into the hollow shank and syringes agent into the hollow shank to gradually raise the pressure in the hollow shank, and the resilient ring outwardly extends to open the multiple holes in the hollow shank when the pressure value in the hollow shank is greater than a resilient force of the resilient ring.

Further benefits and advantages of the present invention will become apparent after a careful reading of the detailed description with appropriate reference to the accompanying drawings.

### BRIEF DESCRIPTION OF THE DRAWINGS

FIG. 1 is a side plan view of a vascular infusion assembly;

FIG. 2 is an exploded perspective view of an infusion port apparatus of the vascular infusion assembly in FIG. 1 in accordance with the present invention;

FIG. 3A is partially cross-sectional view of the infusion port apparatus of the present invention before feeding agent;

FIG. 3B is a partially cross-sectional view of the infusion port apparatus of the present invention during feeding agent;

FIG. 4A is an partially exploded perspective view of a second embodiment of an infusion port apparatus of the vascular infusion assembly in FIG. 1 in accordance with the present invention;

FIG. 4B is a cross-sectional view of the infusion port apparatus in FIG. 4A;

FIG. 5A is an exploded view of a third embodiment of an infusion port apparatus of the vascular infusion assembly in FIG. 1 in accordance with the present invention; and

FIG. 5B is an operational view of the infusion port apparatus in FIG. 5A.

### DETAILED DESCRIPTION OF THE INVENTION

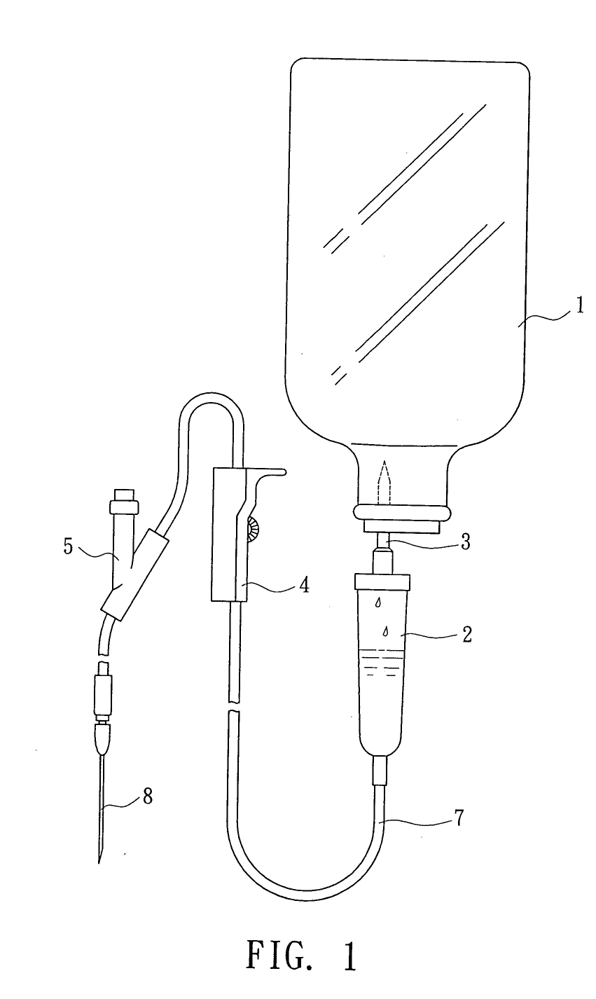

Referring to the drawings and initially to FIG. 1, a vascular infusion assembly usually comprises an agent bottle (1) for containing intravenous infusion agent, a main joint (2) with a insertion (3) inserted into the agent bottle (1), a guide tube (7) connected to the main joint (2) and extending through a controller (4) and a infusion port apparatus (5), and a infusion needle (8) connected to a free end of the guide tube (7). The controller (4) is provided to control the flow rate in the guide tube (7) and the infusion port apparatus (5) is provided for adding extra agent that is not included in the agent bottle (1). However, the agent bottle (1), the main joint (2), the guide tube (7), the controller (4) and the infusion needle (8) are not included in the scope of the present invention so that we do not described in detail hereinafter.

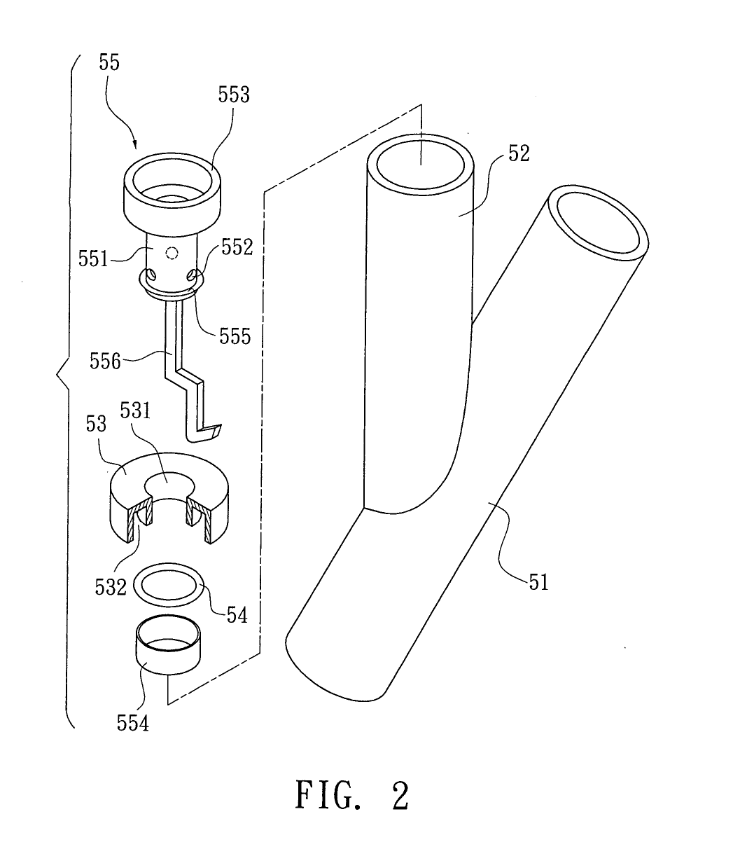

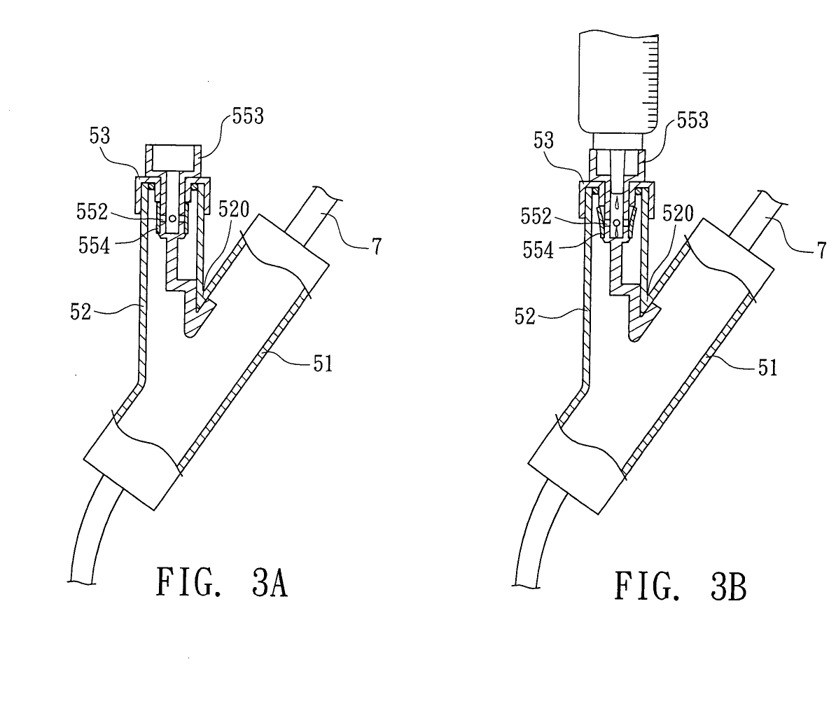

The infusion port apparatus (5) in accordance with the present invention, as shown in FIGS. 2 and 3, comprises a substantially Y-shapes hollow body (50) including a first post (51) adapted to communicate with the guide post (7), and a second post (52) extending from the first post (51) and forming a corner (520) between the first post (51) and the second post (52). The second post (52) communicates with the first post (51) for guiding the agent into the guide tube (7).

An end piece (53) is mounted to a free end of the second post (52). The end piece (53) includes a through hole (531) centrally defined therein and an annular groove (532) defined in a bottom of the end piece (53). An O-ring (54) is received in the annular groove (532). The O-ring (54) respectively abuts against an inner periphery of the second post (52) and an inner side of the annular groove (532) to provide an airtight effect between the end piece (53) and the second post (52).

A feeder (55) mounted to the end piece (53). The feeder (55) includes a hollow shank (551) extending through the through hole (531), and partially and airtightly received in the through hole (531) in the end piece (53). The hollow shank (551) has a close end inserted into the second post (52) and an open end opposite to the close end of the hollow shank (551). The hollow shank (551) includes multiple holes (552) defined therein near the close end of the hollow shank (551) and radially communicating with an inner periphery of the hollow shank (551). An enlarged portion (553) is formed on the open end of the hollow shank (551) to prevent the hollow shank (551) from being overly inserted into the second post (52). A resilient ring (554) is mounted around the hollow shank (551) to selectively close the multiple holes (552) in the hollow shank (551). A protrusion (555) radially outwardly extends from the close end of the hollow shank (551) for supporting the resilient ring (554). A hook (556) longitudinally extends from the close end of the hollow shank (551) and buckles the corner (520) to prevent the feeder (55) from detaching from the second post (52).

With reference to FIGS. 3A, 3B and 3C, when feeding agent into the vascular infusion assembly, a needle hub of a syringe (not numbered) is partially and airtightly received in the open end of the hollow shank (551) and partially received in the hollow shank (551). The pressure in the hollow shank (551) is gradually raised when the agent in syringed into the hollow shank (551). The agent in the hollow shank (551) will outwardly push the resilient ring (554) and flows into the second post (52) via the multiple holes (552) in the hollow shank (551) when the pressure value in the hollow shank (551) is greater than the resilient force of the resilient ring (554). The resilient ring (554) immediately closes the multiple holes (552) when the pressure in the hollow shank (551) is released.

As described above, a sharp needle of the syringe is unnecessary to the present invention so that the infusion port apparatus in accordance with the present invention can prevent the medical personnel and the patient from a suddenly prick due to the sharp needle of the syringe and prevent the medical personnel and the patient from a unexpected infection.

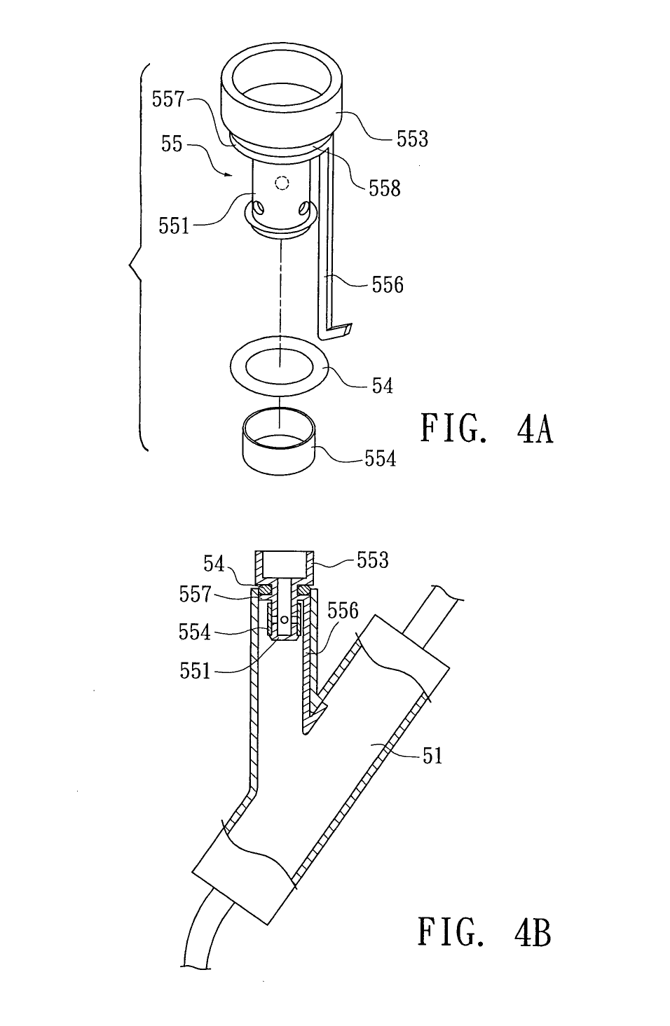

With reference to FIGS. 4A and 4B that show a second embodiment of the infusion port apparatus in accordance with the present invention, the feeder (55) a shoulder (557) radially outwardly extends from the outer periphery of the hollow shank (551) near the enlarged portion (553) to define a groove (558) between the enlarged portion (553) and the shoulder (557). The O-ring (54) is received in the groove (558) and the hook (556) extends from a bottom of the shoulder (557). The effect of the second embodiment is the same as that of the first embodiment of the present invention that is described hereinbefore.

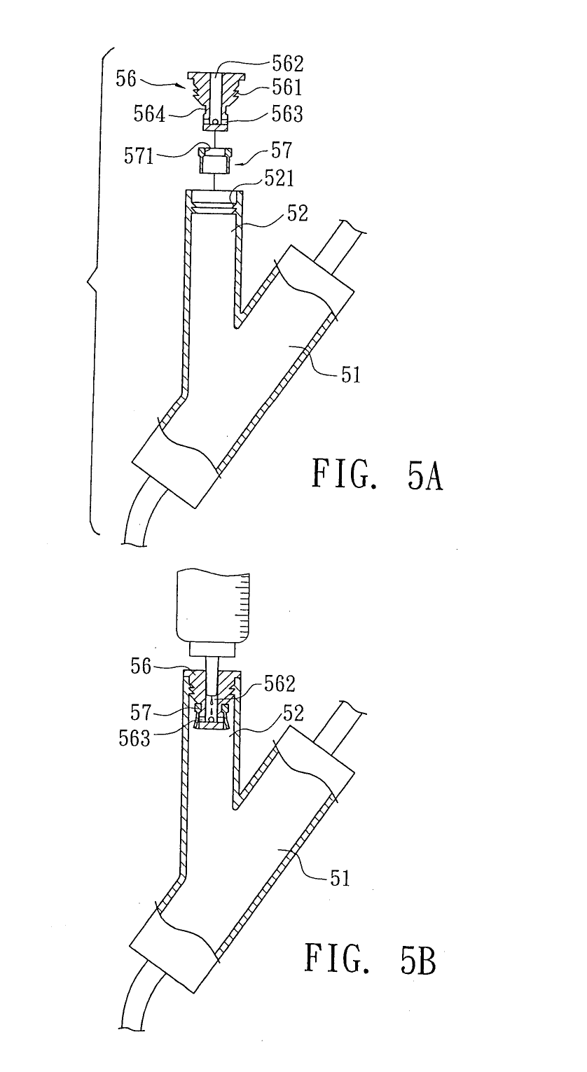

With reference to FIG. 5A that shows a third embodiment of the infusion port apparatus in accordance with the present invention, the second post (52) includes multiple ratchet (521) formed on the inner periphery of the second post (52) near the free end of the second post (52). A feeder (56) is inserted into the free end of the second post (52) and securely received in the second post (52). The feeder (56) includes multiple ratchets (561) outwardly extending from an outer periphery of he feeder (56) and engaged to the ratchet (521) to prevent the feeder (56) form detaching from the second post (52). A blind hole (562) is longitudinally defined in the feeder (56) and extends toward a lower end of the feeder (56). Multiple holes (563) are defined in the outer periphery of the feeder (56) and each laterally communicates with the blind hole (562). An annular groove (564) is defined in the outer periphery of the feeder (56). A resilient ring (57) is mounted around the feeder (56) to selectively close the multiple holes (563). The resilient ring (57) has an annular rib (571) radially extending from an inner periphery of the resilient ring (57) and received in the annular groove (564) to prevent the resilient ring (57) from detaching from the feeder (56).

With reference to FIG. 5B, the operating method and the effect of the third embodiment of the present invention are the same as that of the first embodiment of the present invention that has been described hereinbefore.

Although the invention has been explained in relation to its preferred embodiment, it is to be understood that many other possible modifications and variations can be made without departing from the spirit and scope of the invention as hereinafter claimed.

What is claimed is:

1. An infusion port apparatus of a vascular infusion assembly, comprising:
a first post adapted to longitudinally communicate with a guide post of the vascular infusion assembly;
a second post extending from the first post to form a corner and communicating with the first post; and
a feeder partially airtightly received in the second post, the feeder including:
a hollow shank having a first end extending into and received in the second post, and a second end opposite to the first end of the hollow shank;
multiple holes defined in the hollow shank near the first end of the hollow shank and laterally communicating with an inner periphery of the hollow shank; and
a resilient ring mounted around the hollow shank to selectively close the multiple holes in the hollow shank;
wherein a needle hub of a syringe is inserted into the hollow shank and syringes agent into the hollow shank to gradually raise the pressure in the hollow shank, and the resilient ring outwardly extends to open the multiple holes in the hollow shank when the pressure value in the hollow shank is greater than a resilient force of the resilient ring.

2. The infusion port apparatus as claimed in claim 1, wherein the feeder comprises an enlarged portion formed on the second end of the hollow shank to prevent the feeder from being overly inserted into the second post.

3. The infusion port apparatus as claimed in claim 1, wherein the hollow shank comprises a protrusion radially outwardly extending from the first end of the hollow shank for supporting the resilient ring.

4. The infusion port apparatus as claimed in claim 2, wherein the hollow shank comprises a protrusion radially outwardly extending from the first end of the hollow shank for supporting the resilient ring.

5. The infusion port apparatus as claimed in claim 4, wherein the hollow shank has a shoulder radially outwardly extending therefrom near the enlarged portion to define a groove between the enlarged portion and the shoulder, an O-ring mounted in the groove and airtightly abutting an inner periphery of the second post.

6. The infusion port apparatus as claimed in claim 5, wherein the feeder comprises a hook extending from a bottom of the shoulder and buckled to the corner to prevent the feeder from detaching from the second post.

7. The infusion port apparatus as claimed in claim 4, wherein the feeder comprises an end piece mounted to the free end of the second post and the hollow shank extending through and partially airtightly received in the end piece, an annular groove defined in a bottom of the end piece and an O-ring received in the annular groove, the O-ring respectively abutting against an inner periphery of the second post and an inner side of the annular groove to provide an airtight effect between the end piece and the second post.

8. An infusion port apparatus of a vascular infusion assembly, comprising:
a first post adapted to longitudinally communicate with a guide post of the vascular infusion assembly;
a second post extending from the first post to form a corner and communicating with the first post; and
a feeder partially airtightly received in the end piece, the feeder including:
a blind hole longitudinally defined in the feeder;
multiple hole defined in the feeder and laterally with the blind hole; and
a resilient ring mounted around the feeder to selectively close the multiple holes in the feeder;
wherein a needle hub of a syringe is inserted into the blind hole and syringes agent into the hollow shank to gradually raise the pressure in the blind hole, and the resilient ring outwardly extends to open the multiple holes in the feeder when the pressure value in the blind hole is greater than a resilient force of the resilient ring.

9. The infusion port apparatus as claimed in claim 8, wherein the second post comprises multiple ratchets formed on an inner periphery of the second post and the feeder comprises multiple ratchet formed on an outer periphery of the feeder, the multiple ratchets of the feeder engaged to the multiple ratchets of the second post to prevent the feeder from detaching from the second post.

10. The infusion port apparatus as claimed in claim 8, wherein the feeder comprises an annular groove defined in an outer periphery thereof and the resilient ring has an annular rib radially extending from an inner periphery of the resilient ring, the annular rib of the resilient ring received in the annular groove to prevent the resilient ring from detaching from the feeder.

11. The infusion port apparatus as claimed in claim 9, wherein the feeder comprises an annular groove defined in the outer periphery thereof and the resilient ring has an annular rib radially extending from an inner periphery of the resilient ring, the annular rib of the resilient ring received in the annular groove to prevent the resilient ring from detaching from the feeder.
